(19) United States
(12) Patent Application Publication
(10) Pub. No.: US 2002/0001623 A1
(43) Pub. Date: Jan. 3, 2002

(54) Organism-compatible materials with combined extra-cellular matrices, extracellular-matrix preparations, and production methods

(76) Inventors: Kikuji Yamashita, Myozai-gun (JP); Hidemi Ukai, Yachiyo-shi (JP); Murakami Kouichi, Chiba-shi (JP)

Correspondence Address: KUBOVCIK & KUBOVCIK, SUITE 710, 900 17TH STREET NW, WASHINGTON, DC, 20006

(21) Appl. No.: 09/895,400

(22) Filed: Jul. 2, 2001

(30) Foreign Application Priority Data
Jul. 3, 2000 (JP) 2000-200610

(51) Int. Cl. A61K 35/00; A61K 35/12; A61K 35/32; A61K 35/36

(52) U.S. Cl. 424/520; 424/574

(57) ABSTRACT

This invention provides organism-compatible materials with combined extracellular matrices, which have high affinity with cells of organisms and are capable of hastening cell differentiation, and their production methods. An organism-compatible material with combined extracellular matrices is produced by letting cells of a region of an organism, to which the material is to be applied, form extracellular matrices on a calcification layer formed on a base. When it is applied to the region of the organism, it causes the migration, proliferation, and differentiation of necessary cells to reproduce a necessary tissue quickly.

### BACKGROUND OF THE INVENTION

The present invention relates to organism-compatible materials with combined extracellular matrices, extracellular-matrix preparations, and their production methods. The materials are provided with extracellular matrices in vitro in advance, have high affinity with organisms, are capable of inducing tissues, and can be used to make artificial roots of teeth (oral implants), joints, etc. The preparations are injected as preparations for injection into or applied as ointments to impaired regions of organisms to reproduce necessary tissues quickly.

Metals, ceramics, polymers, etc. have been used as materials for functions of organisms in medical treatment so far. Their demands are increasing for the restoration of lost parts of skin, cartilage, bones, etc. due to accidents and diseases in orthopedics and dentistry.

In dentistry in particular, titanium and titanium alloys, which have an excellent anti-corrosion property, have recently come to be use as materials for functions of bones and teeth.

Because titanium and titanium alloys have poor affinity with bone cells, the former will not attach to the latter easily; accordingly various method of treating the surfaces of the metals have been developed to improve their affinity with bone cells.

For example, hydroxyapatite, the main constituent element of bones, is sprayed on, or ions are implanted into, the surfaces of titanium and titanium alloys to improve their affinity with bone cells.

On the other hand, researches are being made after methods of fixing collagen, gelatin, RGD peptides, etc. on the surfaces of titanium and titanium alloys to improve their affinity with bone cells.

Although hydroxyapatite overlaid on the surfaces of titanium and titanium alloys improves their affinity with bone cells, the strength of the interfaces between the metals and the hydroxyapatite is not strong enough.

In case of the fixation of organic compounds such as tropocollagen extracted as collagen on titanium and titanium alloys, a network of tropocollagen can be formed, but fibers cannot be formed; therefore cell differentiation cannot be hastened enough when the metals are applied to organisms.

Besides, hoped for are preparations for injection and ointments which hasten the restoration of affected tissues of organisms.

An object of the present invention is to provide organism-compatible materials with combined extracellular matrices and their production methods, the materials being free of the above defects, having excellent affinity with cells of organisms, and being capable of hastening enough cell differentiation.

Another object of the present invention is to provide extracellular-matrix preparations for injection and ointments capable of restoring affected parts of organisms quickly and their production methods.

In my research and development of the organism-compatible materials with combined extracellular matrices and the extracellular-matrix preparations, I first studied what reaction, changes, and other phenomena titanium, the most prospective material in dentistry, would cause in the tissues of organisms.

If a piece of titanium is buried in a bone tissue, osteoblasts come in contact with the surface of the piece of titanium; therefore I experimented with culturing osteoblasts on titanium plates.

Phosphoric acid was easily absorbed to the surfaces of the titanium plates to produce hydroxyapatite. Thus a calcification layer was formed on the surface of each titanium plate. Besides, osteoblasts formed extracellular matrices on the calcification layer of each titanium plate.

Although extracellular matrices are known to exist as clear, distinct structures in the space among many cells in most tissues of an organism, it was not known that extracellular matrices could be formed between titanium and osteoblasts cultured on the titanium. In the present experiment, only the osteoblasts could be removed from a group of titanium plates after culturing them on the titanium plates to reveal the existence of the extracellular matrices on the titanium plates.

Osteoblasts secreted tropocollagen and non-collagen protein, which are main constituent elements of extracellular matrices, to form extracellular matrices. Extracellular matrices formed mainly from fibrous collagen were observed not only in the vicinity of each cell but also in areas at small distances from said cell.

One end of each extracellular matrix was joined to the titanium plate and the other end was connected to an osteoblast through integrin receptors, etc. As the calcification progressed on the titanium plate, the fibrous collagen of the former end of said extracellular matrix was buried in the calcification layer and the said extracellular matrix was joined to the titanium plate firmly.

In other words, the osteoblasts (if not removed from extracellular matrices) anchored through the extracellular matrices to the titanium plate, or extracellular matrices themselves anchored to the titanium plate.

When the titanium plates of the above group, after removal of the osteoblasts, were applied to organisms, the extracellular matrices on the titanium plates caused the migration, proliferation, and differentiation of necessary cells to reproduce necessary tissues.

The titanium plates with extracellular matrices of the other group were decalcified to obtain suspension of extracellular matrices alone, which was concentrated. New titanium plates were put in the concentrated suspension, and extracellular matrices anchored to the titanium plates. Thus it was ascertained that extracellular matrices could be anchored onto pieces of titanium in complex shapes which posed difficulty in culturing cells directly on their surfaces.

Some of the suspension of extracellular matrices alone was concentrated by dialysis and sterilized to produce a preparation for injection capable of reproducing necessary tissues quickly. Some of the suspension was concentrated and a base was added to the concentrated suspen sion to prepare an ointment capable of reproducing necessary tissues quickly.

### SUMMARY OF THE INVENTION

The concrete composition of the present invention completed based on the results of the above experiment is as follows.

According to the first aspect of this invention, there is provided an organism-compatible material with combined extracellular matrices comprising a base made of a material for organisms, a calcification layer formed on the base, and extracellular matrices formed on the layer by cells of a region of an organism to which the organism-compatible material with combined extracellular matrices is to be applied.

The advantage offered by the first aspect of the invention is mainly as follows. Because cells are taken from a region of an organism to which the material is to be applied and forms extracellular matrices on a calcification layer formed on a base, the extracellular matrices cause the migration, proliferation, and differentiation of cells of the necessary tissue to reproduce the tissue quickly when the material is applied to the region of the organism.

According to the second aspect of this invention, there is provided the organism-compatible material with combined extracellular matrices according to the first aspect of which the base is of titanium, a titanium alloy, or a calcium-phosphate compound such as hydroxyapatite, or a piece of glass, a piece of a polymer or a ceramic overlaid with titanium, a titanium alloy, or a calcium-phosphate compound such as hydroxyapatite.

The advantage offered by the second aspect of the invention is mainly as follows. Because the extracellular matrices can be anchored onto a base which can be of titanium, a titanium alloy, or a calcium-phosphate compound, or a piece of glass, a piece of a polymer or a ceramic overlaid with titanium, a titanium alloy, or a calcium-phosphate compound, the material has wide applicability to organisms.

According to the third aspect of this invention, there is provided the organism-compatible material with combined extracellular matrices according to the first or second aspect, wherein cells to be used are osteoblasts, chondroblasts, tendon cells, vascular endothelial cells, epithelial cells, connective tissue cells, or glia cells.

The advantage offered by the third aspect of the invention is mainly as follows. Because cells to be used are osteoblasts, chondroblasts, tendon cells, vascular endothelial cells, epithelial cells, connective tissue cells, or glia cells, (i) the material by osteoblasts can be used as artificial bones, joints, roots of teeth (oral implants), etc., (ii) the material by chondroblasts can be used as artificial cartilage, (iii) the material by tendon cells can be implanted into ruptures of tendons, (iv) the material by vascular endothelial cells can be implanted in lost parts of blood vessels, (v) the material by epithelial cells of internal organs can be implanted into organs, (vi) the material by connective tissue cells can be used as artificial skin, and (vii) the material by glia cells can be applied to or implanted into the brain, to reproduce necessary tissues quickly.

According to the fourth aspect of this invention, there is provided the organism-compatible material with combined extracellular matrices according to the first, second, or third aspect which the cells used are not removed from.

The advantage offered by the fourth aspect of the invention is mainly as follows. Because it is unnecessary to remove the cells used, the material is simple and easy to produce. Because the cells used are left as they are on the material, the material has high affinity with the organism.

According to the fifth aspect of this invention, there is provided a production method of an organism-compatible material with combined extracellular matrices, wherein cells of a region of an organism, to which the material is to be applied, are cultured on a base made of titanium or a titanium alloy in a culture solution and, thereby, extracellular matrices are formed between a calcification layer formed on the base and the cells.

The advantage offered by the fifth aspect of the invention is mainly as follows. Because the extracellular matrices are anchored through the calcification layer to the base of titanium or a titanium alloy and the cells used are left on the extracellular matrices, the material is simple and easy to produce and has high affinity with the organism.

According to the sixth aspect of this invention, there is provided a production method of an organism-compatible material with combined extracellular matrices, comprising the steps of (i) culturing cells of a region of an organism, to which the material is to be applied, on a base made of titanium or a titanium alloy in a culture solution to form extracellular matrices between a calcification layer formed on the base and the cells and (ii) removing the cells.

The advantage offered by the sixth aspect of the invention is mainly as follows. Because extracellular matrices can be anchored through a calcification layer to a base of titanium or a titanium alloy, the extracellular matrices cause the migration, proliferation, and differentiation of cells of the necessary tissue to reproduce the tissue quickly when the material is applied to the region of the organism.

According to the seventh aspect of this invention, there is provided the production method of an organism-compatible material with combined extracellular matrices according to the fifth or sixth aspect, wherein the base is a piece of glass, a piece of a polymer, or a ceramic overlaid with titanium or a titanium alloy.

The advantage offered by the seventh aspect of the invention is mainly as follows. Because extracellular matrices can be anchored onto titanium or a titanium alloy overlaid on any of a piece of glass, a piece of a polymer, and a ceramic, the material has wide applicability to organisms.

According to the eighth aspect of this invention, there is provided the production method of an organism-compatible material with combined extracellular matrices according to the fifth, sixth, or seventh aspect, wherein a calcification layer is formed on a surface of the base in a culture solution in advance.

The advantage offered by the eighth aspect of the invention is mainly as follows. Because a calcification layer is formed on a surface of the base in advance, the material can be produced in a short time.

According to the ninth aspect of this invention, there is provided an production method of an organism-compatible material with combined extracellular matrices comprising the steps of (i) culturing cells of a region of an organism, to which the material is to be applied, on a base of titanium or a titanium alloy in a culture solution to form extracellular matrices between a calcification layer formed on the base and the cells, (ii) removing the cells, (iii) decalcifying the base with the calcification layer and the extracellular matrices to obtain suspension of the extracellular matrices, (iv) concentrating the suspension, and (v) combining the extracellular matrices in the concentrated suspension with another base made of titanium or a titanium alloy.

The advantage offered by the ninth aspect of the invention is mainly as follows. Organism-compatible materials with combined extracellular matrices in complex shapes can be produced by anchoring extracellular matrices to bases in complex shapes in suspension of extracellular matrices, and hence the materials have wide applicability to organisms.

According to the tenth aspect of this invention, there is provided an extracellular-matrix preparation for injection which is prepared from extracellular matrices formed by cells of a region of an organism, into which the preparation is to be injected, by concentrating and processing the extracellular matrices.

The advantage offered by the tenth aspect of the invention is mainly as follows. Because the preparation is prepared from the extracellular matrices formed by cells of a region of an organism into which the preparation is to be injected, the preparation is capable of reproducing the necessary tissue quickly when it is injected into the region of the organism.

According to the eleventh aspect of this invention, there is provided an extracellular-matrix ointment which is prepared from concentrated fluid of extracellular matrices formed by cells of a region of an organism, to which the ointment is to be applied, and an ointment base.

The advantage offered by the eleventh aspect of the invention is mainly as follows. Because the ointment is prepared from the extracellular matrices formed by cells of a region of an organism to which the ointment is to be applied, the ointment is capable of reproducing the necessary tissue quickly when it is applied to the region of the organism.

According to the twelfth aspect of this invention, there is provided an production method of an extracellular-matrix preparation for injection comprising the steps of (i) culturing cells of a region of an organism, into which the preparation is to be injected, on a base of titanium or a titanium alloy in a culture solution to form extracellular matrices between a calcification layer formed on the base and the cells, (ii) removing the cells, (iii) decalcifying the base with the calcification layer and the extracellular matrices to obtain suspension of the extracellular matrices, (iv) concentrating the suspension by dialysis, (v) sterilizing the concentrated suspension, and (vi) preparing the preparation for injection from the concentrated suspension.

The advantage offered by the twelfth aspect of the invention is mainly that the preparation for injection prepared from cells of a region of an organism into which the preparation is to be injected is capable of reproducing the necessary tissue quickly.

According to the thirteenth aspect of this invention, there is provided an production method of an extracellular-matrix ointment comprising the steps of (i) culturing cells of a region of an organism, to which the ointment is to be applied, on a base of titanium or a titanium alloy in a culture solution to form extracellular matrices between a calcification layer formed on the base and the cells, (ii) removing the cells, (iii) decalcifying the base with the calcification layer and the extracellular matrices to obtain suspension of the extracellular matrices, (iv) concentrating the suspension and (v) adding an ointment base to the concentrated suspension to prepare the ointment from the concentrated suspension.

The advantage offered by the thirteenth aspect of the invention is mainly that the ointment prepared from cells of a region of an organism to which the ointment is to be applied is capable of reproducing the necessary tissue quickly.

### BRIEF DESCRIPTION OF THE DRAWINGS

The features and advantages of the present invention will become more clearly appreciated from the following description in conjunction with the accompanying drawings, in which:

FIG. 1 is an illustration of an embodiment of organism compatible material with combined extracellular matrices of the present invention, the base of the material being of titanium;

FIG. 2 is an illustration of another embodiment of organism-compatible material with combined extracellular matrices of the present invention, the base of the material being of titanium;

FIGS. 3(a) and (b) are photos, taken by an electron microscope, of a calcification layer formed on a base (a titanium plate) of an organism-compatible material with combined extracellular matrices according to the present invention;

FIGS. 4(a) and (b) are photos, taken by an electron microscope after five days of culture, of extracellular matrices formed on a base (a titanium plate) of an organism-compatible material with combined extracellular matrices according to the present invention; and

FIGS. 5(a) and (b) are photos, taken by an electron microscope after seven days of culture, of extracellular matrices formed on a base (a titanium plate) of an organism-compatible material with combined extracellular matrices according to the present invention.

### DETAILED DESCRIPTION OF THE PREFERRED EMBODIMENT

Referring to the drawings, a preferred embodiment of organism-compatible material with combined extracellular matrices and its production method of the present invention will now be described.

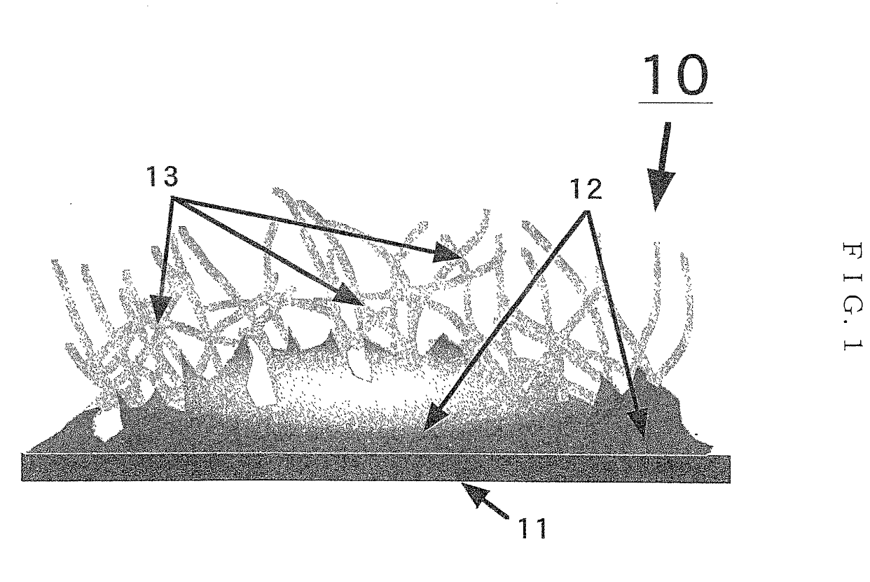

In FIG. 1, reference numeral 10 is an organism-compatible material with combined extracellular matrices; 11, a titanium base; 12, a calcification layer; and 13, extracellular matrices. As shown in the figure, the calcification layer 12 is formed on the base 11. Each matrix 13 is formed with its one end buried in and joined to, or combined with, (i.e., anchored in) the calcification layer 12.

The extracellular matrices 13 of the organism-compatible material with combined extracellular matrices 10 are formed by cells of supporting or epithelial tissues of a region of an organism to which the material 10 is to be applied. Such a region may be a bone, a periodontal membrane, a joint, a tendon, shin, a blood vessel, an internal organ, or the brain.

If such a region is a bone or a periodontium, extracellular matrices 13 are formed by osteoblasts or cells of a periodontal membrane, as the case may be.

If such a region is (i) cartilage of a joint, (ii) a rupture of a tendon, (iii) skin, (iv) a vessel, (v) an organ, or (vi) the brain, extracellular matrices 13 are formed by (i) chondroblasts, (ii) tendon cells, (iii) connective tissue cells, (iv) vascular endothelial cells, (v) epithelial cells, or (vi) neurogliacytes, as the case may be.

The main constituent elements of such extracellular matrices 13 are fibrous collagen and non-collagen protein. Each of the cells (for example, osteoblasts) to form extracellular matrices 13 for a supporting tissue secretes fibrous collagen to form extracellular matrices 13. The extracellular matrices 13 are formed from not only the vicinity of said cell but also areas at small distances from said cell.

When the organism-compatible material with combined extracellular matrices 10 is applied to an organism, the extracellular matrices 13 formed by each cell cause the migration, proliferation, and differentiation of necessary cells to reproduce a necessary tissue.

A production method of the organism-compatible material with combined extracellular matrices 10 will next be described.

(1) A titanium base 11 is put into a culture solution of cells (for example, osteoblasts) 14 of a region of an organism. Phosphoric acid is easily absorbed to the surface of the base 11 to produce hydroxyapatite. Thus a calcification layer 12 is formed.

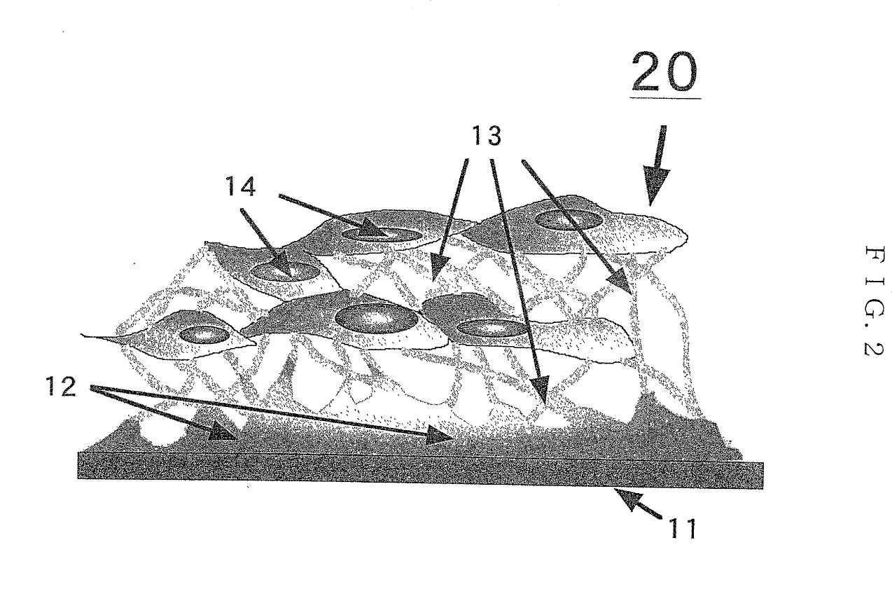

(2) The cells (for example, cells of supporting tissues such as osteoblasts) 14 of the region of the organism are cultured on the calcification layer 12 of the base 11 or on the base 11. In case of the culture on the calcification layer 12, extracellular matrices 13 are formed on the calcification layer 12 by the cells 14 as shown in FIG. 2. In case of the culture on the base 11, a calcification layer 12 is formed as described in the above paragraph (1) and, then, extracellular matrices 13 are formed on the calcification layer 12 by the cells 14 as shown in FIG. 2. In either case, one end of each extracellular matrix 13 is joined to the base 11 and the other end is connected to a cell 14 through integrin receptors, etc. As the calcification progresses on the base 11, the fibrous collagen of the former end of said extracellular matrix 13 is buried in the calcification layer 12 and the said extracellular matrix 13 is joined to the base 11 firmly. Thus said extracellular matrix 13 is anchored to the base 11.

(3) After forming the extracellular matrices 13 on the calcification layer 12 on the base 11 by culturing the cells 14 on the base 11, the cells 14 are removed. The cells 14 can be removed, for example, by combining drying and dynamic detachment. To be concrete, the base 11 with the calcification layer 12, the extracellular matrices 13, and the cells 14 are dehydrated and dried to the critical point, and the cells 14 are physically removed by using adhesive tape. Thus an organism-compatible material with combined extracellular matrices 10 can be obtained.

In accordance with the above production method, the organism-compatible material with combined extracellular matrices 10 can be produced easily. The organism-compatible material with combined extracellular matrices 10 applied to a region of an organism causes the migration, proliferation, and differentiation of cells to reproduce a necessary tissue quickly.

Besides, because the cells 14 are removed from it, the organism-compatible material with combined extracellular matrices 10 has excellent affinity to organisms, causes few problems about immunity, and can be applied to wide areas.

To culture the cells of different regions of an organism, different culture solutions are necessary. Osteoblasts require α-MEM and 10% fetal calf serum; chondroblasts, α-MEM and 0.5% fetal calf serum; tendon cells, DMEM and 10% fetal calf serum; connective tissue cells, DMEM and 10% fetal calf serum; vascular endothelial cells, DMEM and 20% fetal calf serum; epithelial cells, MEM and 20% fetal calf serum; and glia cells, HAMF12, glial growth factor, insulin, triiodthyronine, etc.

The base may be of titanium, a titanium alloy, a calcium-phosphate compound such as hydroxyapatite, calcium, phosphoric acid, or calcium and phosphoric acid, or the base may be a piece of glass, a piece of a polymer, or a ceramic which is irradiated with titanium plasma. Any other materials can be used as the substratum of the base so long as they are applicable to organisms and allow the formation of a calcification layer on them.

Another embodiment of organism-compatible material with combined extracellular matrices and its production method of the present invention will next be described. This material and its production method are partly the same as the above organism-compatible material with combined extracellular matrices 10 and its production method; accordingly description of the same parts will be omitted here.

As shown in FIG. 2, cells (for example, cells of supporting tissues such as osteoblasts) 14 of a region of an organism are cultured on a base 11 to form extracellular matrices 13, and the cells 14 are left as they are on the organism-compatible material with combined extracellular matrices 20.

When the organism-compatible material with combined extracellular matrices 20 with the cells 14 is applied to an organism, it causes the migration, proliferation, and differentiation of necessary cells to reproduce a necessary tissue.

Because the cells 14 are not removed, the organism compatible material with combined extracellular matrices 20 is easy to produce. Besides, if the material 20 is applied to the same patient whom the cells 14 were taken from, it shows excellent affinity with the patient, causing no problem about immunity.

Another embodiment of production method of an organism compatible material with combined extracellular matrices of the present invention will next be described. This production method is partly the same as the above production method; accordingly description of the same part will be omitted here.

In accordance with this production method, a base is put in a concentrated suspension of extracellular matrices and the extracellular matrices anchor onto the base. This method is suitable to such a case as the shape of the base is complex and it is difficult to culture the cells of a region of an organism directly on the surface of the base.

In accordance with this production method, cells 14 are cultured on a base 11, such as titanium, in the shape of a flat plate or in any other shape suitable for the culture of the cells 14 to form extracellular matrices 13 and then the cells 14 are removed in accordance with the above steps (1) to (3) for the organism-compatible material with combined extracellular matrices 10. Accordingly, an organism-compatible material with combined extracellular matrices is obtained, the extracellular matrices 13 anchoring onto the base 11 though a calcification layer 12.

(4) Next, the base 11 is decalcified to obtain a suspension of the extracellular matrices 13 alone, and the suspension is concentrated.

(5) Another base such as titanium in a complex shape suitable to organisms is put in the concentrated suspension of extracellular matrices 13. The extracellular matrices 13 anchor onto the surface of the base 11 through a calcification layer 12. Thus an organism-compatible material with combined extracellular matrices 10 is obtained.

In accordance with this production method of organism compatible materials with combined extracellular matrices, it is unnecessary to culture cells 14 directly on bases in complex shapes; therefore organism-compatible materials with combined extracellular matrices in complex shapes can be made for wide application to organisms.

An embodiment of extracellular-matrix preparation and its production method of the present invention will next be described. This production method is partly the same as the above production methods of organism-compatible materials with combined extracellular matrices; accordingly description of the same part will be omitted here.

The extracellular-matrix preparation is a preparation for injection prepared from concentrated suspension of extracellular matrices. The preparation for injection is suitable for direct injection to regions of organisms to reproduce tissues in the regions.

In accordance with this production method, cells 14 are cultured on a base 11, such as titanium, in the shape of a flat plate or in any other shape suitable for the culture of the cells 14 to form extracellular matrices 13, which anchor onto the base 11 though a calcification layer 12, and then the cells 14 are removed in accordance with the above steps (1) to (3) for an organism-compatible material with combined extracellular matrices 10.

(4) Next, the base 11 is decalcified to obtain suspension of the extracellular matrices 13 alone, and the suspension is concentrated by dialysis.

(5) The concentrated suspension of extracellular matrices 13 is sterilized and a preparation for injection is prepared from the suspension.

When the preparation for injection is injected into a region of an organism, the extracellular matrices 13 formed by each cell cause the migration, proliferation, and differentiation of necessary cells and the accretionary formation of extracellular matrices for a necessary tissue in the region.

Accordingly the necessary tissue in the region can be reproduced quickly.

In accordance with the above production method, preparations for injection which reproduce necessary tissues quickly and have wide applicability to organisms can be prepared from extracellular matrices.

Another embodiment of extracellular-matrix preparation and its production method of the present invention will next be described. This preparation and its production method are partly the same as the above extracellular-matrix preparation and its production method; accordingly description of the same parts will be omitted here.

This extracellular-matrix preparation is an ointment prepared from concentrated suspension of extracellular matrices, the ointment being suitable for direct application to regions of organisms to reproduce tissues in the regions.

In accordance with this production method, cells 14 are cultured on a base 11, such as titanium, in the shape of a flat plate or in any other shape suitable for the culture of the cells 14 to form extracellular matrices 13, which anchor onto the base 11 though a calcification layer 12, and then the cells 14 are removed in accordance with the above steps (1) to (3) for an organism-compatible material with combined extracellular matrices 10.

(4) Next, the base 11 is decalcified to obtain suspension of the extracellular matrices 13 alone, and the suspension is concentrated.

(5) An ointment base is added to the concentrated suspension of extracellular matrices 13 to prepare an ointment.

When the ointment is applied to a region of an organism, the extracellular matrices 13 formed by each cell cause the migration, proliferation, and differentiation of necessary cells and the accretionary formation of extracellular matrices for a necessary tissue in the region.

Accordingly the necessary tissue in the region can be reproduced quickly.

In accordance with the above production method, ointments which reproduce necessary tissues quickly and have wide applicability to organisms can be prepared from extracellular matrices.

### EXAMPLES

### Example 1

An example of production methods of organism-compatible materials with combined extracellular matrices of the present invention will be described below, but the scope of the present invention is not to be limited to the example.

(1) Titanium plates were prepared as bases, and their surfaces were grinded and polished with waterproof sandpaper of P180, P600, P1500, P2000, and P3000 and fine diamond film (abrasive paper) of P4000.

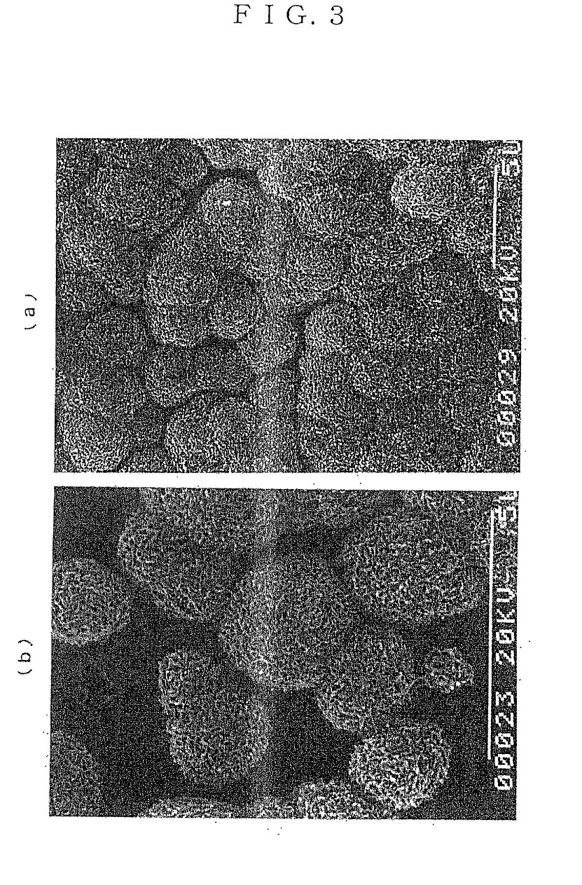

(2) The grinded and polished titanium plates were cultured in a α-MEM (GibcoBRL) culture solution containing 10% fetal calf serum and antibiotics at 37° C. in air containing 5% C0₂ for one to three weeks for preliminary calcification. As shown in FIGS. 3(a) and (b), a calcification deposit is formed on each titanium plate after three weeks of preliminary calcification.

(3) Osteoblasts were suspended and seeded in the culture solution at the rate of 50,000 osteoblasts/ml and cultured under the same conditions in the above paragraph (2).

(4) Some titanium plates were taken out of the culture solution after five days of culture; the other titanium plates, after seven days of culture. Each titanium plate was dehydrated for seven minutes, twice, with each of 50%, 70%, 80%, 90%, 95%, and 100% ethanol solutions.

(5) The titanium plates were dried with a critical-point drier (HCP=2 of Hitachi).

(6) The cell layer on the top of each titanium plate-cum-extracellular matrices was removed with pressure-sensitive adhesive-double coated tape.

Produced through the above steps (1) to (6) were titanium plates to which extracellular matrices were anchored through a calcification layer.

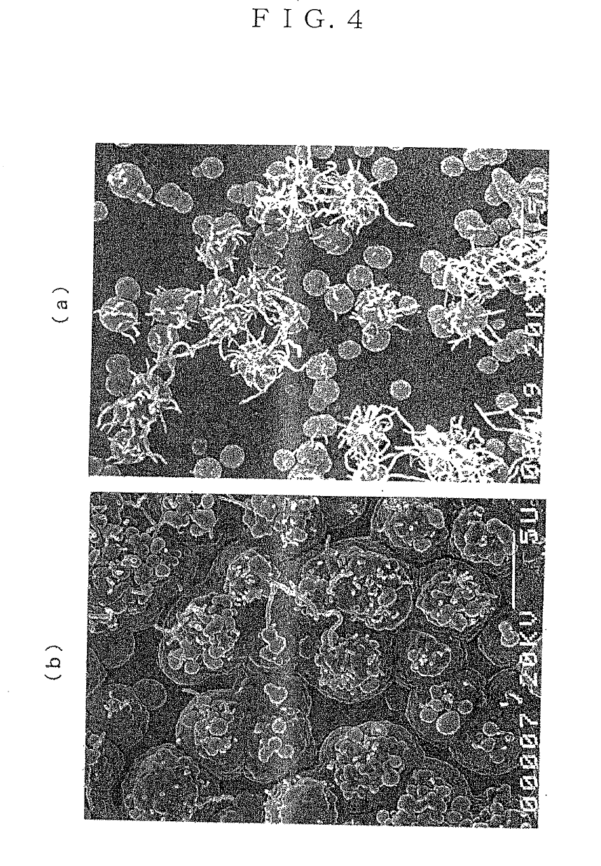

As shown in FIGS. 4(a) and (b), each five-day-cultured organism-compatible material with combined extracellular matrices produced through the above steps has fibers of early extracellular matrices formed on a calcification deposit laid on the titanium plate.

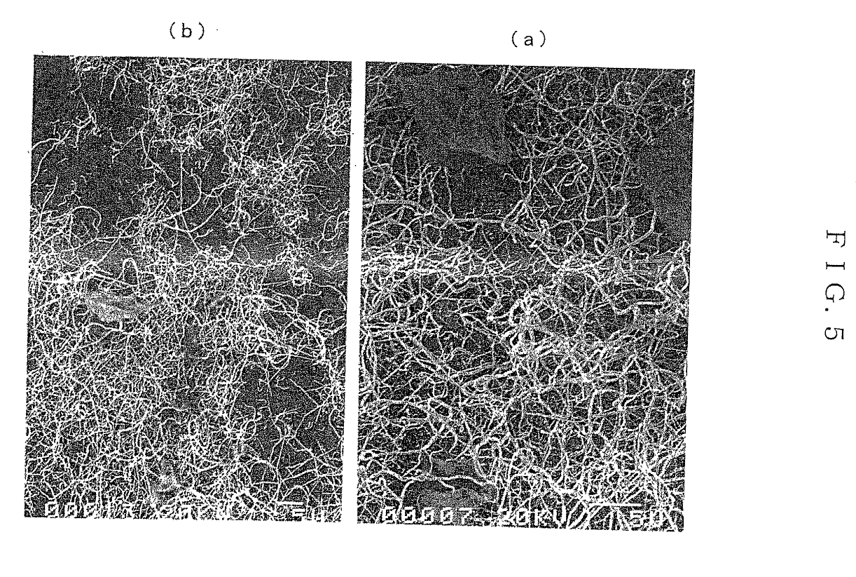

As shown in FIGS. 5(a) and (b), each seven-day-cultured organism-compatible material with combined extracellular matrices produced through the above steps has a network of extracellular matrices formed on a calcification deposit laid on the titanium plate.

Above titanium plates were applied to lost parts of bones of animals to ascertain quick formation of bone tissues.

### Example 2

Examples of organism-compatible materials with combined extracellular matrices of the present invention will be described below, but the scope of the present invention is not to be limited to the examples.

(1) Organism-compatible materials with combined extracellular matrices produced by culturing osteoblasts

(a) Titanium

A titanium plate with combined extracellular matrices produced by culturing osteoblasts may be applied and fixed to a broken bone or lost part of a bone with titanium nails, or a few titanium plates with combined extracellular matrices, one on top of another, may be buried in lost part of a bone in orthopedics and dentistry.

Thin titanium film with combined extracellular matrices produced by culturing osteoblasts may be wrapped around broken part or lost part of a bone in orthopedics and dentistry.

Titanium plates and rods with combined extracellular matrices produced by culturing osteoblasts may be used to make artificial joints and roots of teeth (oral implants) in orthopedics and dentistry.

(b) Calcium Phosphate such as Hydroxyapatite

Calcium phosphate such as hydroxyapatite with combined extracellular matrices produced by culturing osteoblasts may be used as a filler for lost parts of bones in orthopedics and dentistry.

(c) Polymers and other Materials which contain Calcium Phosphate such as Hydroxyapatite or with which Titanium is combined by Plasma Irradiation

Polymer film with combined extracellular matrices produced by culturing osteoblasts may be wrapped around broken or lost parts of bones or a few film sheets one on top of another are buried in broken or lost part of a bone in orthopedics and dentistry. The polymer film may, as GTR or GBR film, be attached to lost parts of bones in dentistry.

(2) Organism-compatible materials with combined extracellular matrices produced by culturing chondroblasts

(a) Calcium Phosphate such as Hydroxyapatite

Calcium phosphate such as hydroxyapatite with combined extracellular matrices produced by culturing chondroblasts may be used as a filler for lost parts of cartilage of joints due to articular rheumatism, etc. and lost parts of permanent cartilage of other regions in orthopedics.

(b) Polymers and other Materials which contain Calcium Phosphate such as Hydroxyapatite or with which Titanium is combined by Plasma Irradiation

Polymer film with combined extracellular matrices produced by culturing chondroblasts may be attached to lost parts of cartilage of joints due to articular rheumatism, etc., lost parts of joint disks of jaw joints, etc., and lost parts of permanent cartilage of other regions, or a few film sheets one on top of another may be buried in such parts in orthopedics.

(3) Organism-compatible materials with combined extracellular matrices produced by culturing tendon cells

(a) Polymers and other materials which contain calcium phosphate such as Hydroxyapatite or with which titanium is combined by plasma irradiation

Polymers with combined extracellular matrices produced by culturing tendon cells may be wrapped around ruptures of tendons and fixed or buried in such parts in orthopedics.

(4) Organism-compatible materials with combined extracellular matrices produced by culturing vascular endothelial cells

(a) Polymers and other materials which contain calcium phosphate such as hydroxyapatite or with which titanium is combined by plasma irradiation

Polymer tubes with combined extracellular matrices produced by culturing vascular endothelial cells may be applied to lost parts of blood vessels as artificial vessels in surgery.

(5) Organism-compatible materials with combined extracellular matrices produced by culturing dermatogenic fibroblasts and periodontal membranes

(a) Polymers and other Materials which contain Calcium Phosphate such as Hydroxyapatite or with which Titanium is combined by Plasma Irradiation

Polymer film with combined extracellular matrices produced by culturing dermatogenic fibroblasts may be applied to lost parts of skin due to skin trouble such as burns and scalds as artificial skin and coria in surgery.

(b) Polymers and other Materials which contain calcium phosphate such as hydroxyapatite or with which Titanium is combined by plasma irradiation

Polymer film with combined extracellular matrices produced by culturing cells of periodontal membranes may be applied to lost parts of periodontal membranes of periodontia as GTR membranes capable of inducing periodontal membranes or buried in such parts in dentistry.

(6) Organism-compatible materials with combined extracellular matrices produced by culturing organogenic connective tissue cells or epithelial cells

Polymer film with combined extracellular matrices produced by culturing hepatogenic epithelial cells may be applied to lost part of the liver due to fatty cirrhosis, hepatic cancer, or the like, or a few sheets of the polymer film one on top of another may be buried in the lost part in surgery. Many polymer films with combined extracellular matrices produced by culturing hepatogenic epithelial cells may be used in vitro as an artificial liver in surgery.

Polymer film with combined extracellular matrices produced by culturing pulmonary epithelial cells may be applied to lost part of a lung due to pneumonia, lung cancer, or the like, or a few sheets of the polymer film one on top of another may be buried in the lost part in surgery.

Polymer tubes with combined extracellular matrices produced by culturing epithelial cells of hollow organs such as enteric canals may be applied to lost parts of hollow organs in surgery.

Polymer film with combined extracellular matrices produced by culturing epithelial cells of other organs may be applied to lost parts of such organs due to inflammatory diseases, cancer, etc., or a few sheets of the polymer film one on top of another may be buried in such parts in surgery.

(7) Organism-compatible materials with combined extracellular matrices produced by culturing glia cells

(a) Polymers and other Materials which contain Calcium Phosphate such as Hydroxyapatite or with which Titanium is combined by Plasma Irradiation

Polymer film with combined extracellular matrices produced by culturing neurogliacytes may be applied to lost or atrophic part of the brain due to cerebral infarction, Alzheimer's disease, or the like, or a few sheets of the polymer film may be buried in the lost or atrophic part in surgery.

(8) Organism-compatible materials with combined extracellular matrices produced by culturing more than two kinds of cells

Extracellular matrices of cells, such as osteoblasts, of more than two kinds of supporting tissues (composite extracellular matrices) can be anchored onto a base.

(a) Titanium

Extracellular matrices are formed on the surface of a titanium plate or bar by culturing osteoblasts, and the osteoblasts are removed.

Next, extracellular matrices are formed on the first layer of extracellular matrices by culturing cells of periodontal membranes, and the cells are removed.

Lastly, extracellular matrices are formed on the second layer of extracellular matrices by culturing, again, osteoblasts, and the osteoblasts are removed.

Such titanium plates and bars with combined composite extracellular matrices may be used to make artificial joints and roots of teeth (oral implants) in orthopedics and dentistry.

When such an artificial joint or an artificial root is applied to an organism, a connective tissue such as a periosteum or a periodontal membrane, as the case may be, is produced around it and bone is reproduced.

(b) Polymers and other Materials which contain Calcium Phosphate such as Hydroxyapatite or with which Titanium is combined by Plasma Irradiation

Polymer film, whose one side is overlaid with extracellular matrices by culturing dermatogenic epitheliums and whose other side is overlaid with extracellular matrices by culturing fibroblasts of coria, may be applied to lost parts of skin due to skin trouble such as burns and scalds as compound artificial skin in surgery.

The compound artificial skin is capable of inducing a corium and an epithelium simultaneously at a lost part of skin.

### Example 3

Examples of extracellular-matrix preparations of the present invention will be described below, but the scope of the present invention is not to be limited to the examples.

(1) Extracellular-matrix preparations prepared from osteoblasts

(a) Preparation for Injection

An extracellular-matrix preparation for injection to be prepared from osteoblasts may be injected into broken part of a bone or under the periosteum of a bone-resorption region to cure the broken part or reproduce bone in the region quickly.

When the extracellular-matrix preparation is injected into boneresorption regions of alveolar bones due to pyorrhea, it induces the restoration of alveolar bones to increase the occlusal force and prevent the loss of teeth.

In the treatment of a lost tooth, the extracellular-matrix preparation may be injected into regions short of ossein to produce ossein in the regions prior to implanting an artificial root in the bone.

(b) Ointment

An extracellular-matrix ointment to be prepared from osteoblasts may, in addition to surgical treatment, be applied to large lost part of a bone due to an accident or a disease such as cancer to hasten the reproduction of bone at the part.

In addition to a flap operation to remove impaired gum and foci of pyorrhea, the extracellular-matrix ointment may be used to reproduce bone quickly and makes it possible to treat the pyorrhea thoroughly.

The extracellular-matrix ointment may be applied to the cavity made by extracting a tooth, prior to implanting an artificial root in the cavity, to quicken the treatment of the wound and produce bone.

(2) Extracellular-matrix preparations prepared from chondroblasts

(a) Preparation for Injection

An extracellular-matrix preparation for injection to be prepared from chondroblasts may be injected into lost parts of cartilage of joints due to articular rheumatism, etc. and impaired parts of permanent cartilage of other regions to reproduce cartilage quickly.

(b) Ointment

An extracellular-matrix ointment to be prepared from chondroblasts may, in addition to surgical treatment, be applied lost parts of cartilage of joints due to severe articular rheumatism, etc. and lost parts of permanent cartilage of other regions to hasten the reproduction of cartilage.

(3) Extracellular-matrix preparations prepared from tendon cells

(a) Preparation for Injection

An extracellular-matrix preparation for injection to be prepared from tendon cells may be injected into an impaired or ruptured Achilles' or other tendon or may be used, in addition to surgical treatment of such a tendon, to hasten its restoration.

(b) Ointment

An extracellular-matrix ointment to be prepared from tendon cells may, in addition to surgical treatment, be applied to a ruptured or otherwise damaged Achilles' or other tendon to hasten its restoration.

(4) Extracellular-matrix preparations prepared from dermatogenic fibroblasts and epithelial cells

(a) Ointments

An extracellular-matrix ointment to be prepared from dermatogenic fibroblasts or epithelial cells may be applied to lost part of skin due to skin trouble such as a burn or a scald or may, in addition to surgical treatment, be applied to the lost part of skin.

The ointment may be applied to slight burns and scalds, scratches, and incised wounds to hasten their healing.

(5) Extracellular-matrix preparations prepared from cells of connective tissues of periodontal membranes

(a) Preparation for Injection

An extracellular-matrix preparation for injection to be prepared from cells of connective tissues of periodontal membranes may be injected into lost parts of periodontal membranes due to dental caries or pyorrhea to hasten the restoration of the periodontal membranes, restore the occlusal force, and prevent the loss of teeth.

(b) Ointment

An extracellular-matrix ointment to be prepared from cells of connective tissues of periodontal membranes may be applied to lost parts of periodontal membranes due to dental caries or pyorrhea to hasten the restoration of the periodontal membranes, restore the occlusal force, and prevent the loss of teeth.

(6) Extracellular-matrix preparations prepared from organogenic connective tissue cells or epithelial cells

(a) Preparations for Injection

An extracellular-matrix preparation for injection to be prepared from hepatogenic connective tissue cells or epithelial cells may be injected into lost part of the liver due to cirrhosis, hepatic cancer, or the like to hasten the restoration of the lost part and the function of the liver.

An extracellular-matrix preparation for injection to be prepared from pneumogenic connective tissue cells or epithelial cells may be injected into lost part of a lung due to pneumonia, lung cancer, or the like to hasten its restoration and the restoration of its function.

Extracellular-matrix preparations for injection to be prepared from other organogenic connective tissue cells or epithelial cells may be injected into lost parts of such organs due to diseases to hasten their restoration and the restoration of their functions.

(b) Ointments

An extracellular-matrix ointment to be prepared from hepatogenic connective tissue cells or epithelial cells may, in addition to surgical treatment, be applied to lost part of the liver due to cirrhosis, hepatic cancer, or the like to hasten the restoration of the lost part and the function of the liver.

An extracellular-matrix ointment to be prepared from pneumogenic connective tissue cells or epithelial cells may, in addition to surgical treatment, be applied to lost part of a lung due to pneumonia, lung cancer, or the like to hasten its restoration and the restoration of its function.

Extracellular-matrix ointments to be prepared from other organogenic connective tissue cells or epithelial cells may, in addition to surgical treatment, be applied to lost parts of such organs due to diseases to hasten their restoration and the restoration of their functions.

(7) Extracellular-matrix preparations prepared from glia cells

(a) Preparation for Injection

An extracellular-matrix preparation for injection to be prepared from glia cells may be administered by spinal puncture into the cerebrospinal fluid of a patient with a disease of the central nervous system such as Alzheimer's disease or slight cerebral infarction to activate his nerve cells and thereby improve his condition.

(b) Ointment

An extracellular-matrix ointment to be prepared from glia cells may, in addition to surgical treatment, be applied to affected parts of a patient with a disease of the central nervous system such as Alzheimer's disease or slight cerebral infarction to activate his nerve cells and thereby improve his condition.

The invention may be embodied in other specific forms without departing from the spirit or essential characteristics thereof. The above embodiment is therefore to be considered in all respects as illustrative and not restrictive, the scope of the invention being indicated by the appended claims rather than by the foregoing description and all changes which come within the meaning and range of equivalency of the claims are therefore intended to be embraced therein.

What is claimed is:

1. An organism-compatible material with combined extracellular matrices comprising a base made of a material for organisms, a calcification layer formed on the base, and extracellular matrices formed on the layer by cells of a region of an organism to which the organism-compatible material with combined extracellular matrices is to be applied

2. An organism-compatible material with combined extracellular matrices as claimed in claim 1 of which the base is of titanium, a titanium alloy, or a calcium-phosphate compound such as hydroxyapatite, or a piece of glass, a piece of a polymer or a ceramic overlaid with titanium, a titanium alloy, or a calcium-phosphate compound such as hydroxyapatite.

3. An organism-compatible material with combined extracellular matrices as claimed in claim 1 or 2, wherein said cells are osteoblasts, chondroblasts, tendon cells, vascular endothelial cells, epithelial cells, connective tissue cells, or glia cells.

4. An organism-compatible material with combined extracellular matrices as claimed in claim 1, 2, or 3 which includes said cells.

5. A production method of an organism-compatible material with combined extracellular matrices, wherein cells of a region of an organism, to which the material is to be applied, are cultured on a base made of titanium or a titanium alloy in a culture solution and, thereby, extracellular matrices are formed between a calcification layer formed on the base and the cells.

6. A production method of an organism-compatible material with combined extracellular matrices, comprising the steps of:
culturing cells of a region of an organism, to which the material is to be applied, on a base made of titanium or a titanium alloy in a culture solution to form extracellular matrices between a calcification layer formed on the base and the cells; and
removing the cells.

7. A production method of an organism-compatible material with combined extracellular matrices as claimed in claim 5 or 6, wherein the base is a piece of glass, a piece of a polymer, or a ceramic overlaid with titanium or a titanium alloy.

8. A production method of an organism-compatible material with combined extracellular matrices as claimed in claim 5, 6, or 7, wherein a calcification layer is formed on a surface of the base in a culture solution in advance.

9. A production method of an organism-compatible material with combined extracellular matrices comprising the steps of:
culturing cells of a region of an organism, to which the material is to be applied, on a base of titanium or a titanium alloy in a culture solution to form extracellular matrices between a calcification layer formed on the base and the cells;
removing the cells;
decalcifying the base with the calcification layer and the extracellular matrices to obtain suspension of the extracellular matrices;
concentrating the suspension; and
combining the extracellular matrices in the concentrated suspension with another base made of titanium or a titanium alloy.

10. An extracellular-matrix preparation for injection which is prepared from extracellular matrices formed by cells of a region of an organism, into which the preparation is to be injected, by concentrating and processing the extracellular matrices.

11. An extracellular-matrix ointment which is prepared from concentrated fluid of extracellular matrices formed by cells of a region of an organism, to which the ointment is to be applied, and an ointment base.

12. A production method of an extracellular-matrix preparation for injection comprising the steps of:
culturing cells of a region of an organism, into which the preparation is to be injected, on a base of titanium or a titanium alloy in a culture solution to form extracellular matrices between a calcification layer formed on the base and the cells;
removing the cells;
decalcifying the base with the calcification layer and the extracellular matrices to obtain suspension of the extracellular matrices;
concentrating the suspension by dialysis;
sterilizing the concentrated suspension; and
preparing the preparation for injection from the concentrated suspension.

13. A production method of an extracellular-matrix ointment comprising the steps of:
culturing cells of a region of an organism, to which the ointment is to be applied, on a base of titanium or a titanium alloy in a culture solution to form extracellular matrices between a calcification layer formed on the base and the cells;
removing the cells;
decalcifying the base with the calcification layer and the extracellular matrices to obtain suspension of the extracellular matrices;
concentrating the suspension; and
adding an ointment base to the concentrated suspension to prepare the ointment from the concentrated suspension.
